(19) United States
(12) Patent Application Publication
(10) Pub. No.: US 2022/0370712 A1
(43) Pub. Date: Nov. 24, 2022

(54) Infusion cannula unit made of plastic

(71) Applicant: RAUMEDIC AG, Münchberg (DE)

(72) Inventors: Björn GLÄSEL, Kirchenlamitz (DE); Tobias FESTEL, Sparneck (DE)

(73) Assignee: RAUMEDIC AG, Münchberg (DE)

(21) Appl. No.: 17/772,938

(22) PCT Filed: Oct. 8, 2020
(86) PCT No.: PCT/EP2020/078217

§ 371 Date: Apr. 28, 2022

(30) Foreign Application Priority Data
Oct. 31, 2019 (DE) 10 2019 216 870.1

(51) Int. Cl. A61M 5/158

(52) U.S. Cl. CPC A61M 5/158; A61M 2005/1587

(57) ABSTRACT

An infusion cannula unit made of plastic has a cannula for insertion in a blood vessel. A housing of the unit has a supply line portion the line axis of which runs at an angle relative to a cannula longitudinal axis, said angle being greater than 10°. The supply line portion is in fluid communication with a lumen of the cannula. The infusion cannula unit has a one-piece design. This results in a unit which is easy to produce and in particular suitable for mass production.

### CROSS-REFERENCES TO RELATED APPLICATIONS

This application claims the priority of German Patent Application, Serial No. DE 10 2019 216 870.1, filed Oct. 31, 2019, the content of which is incorporated herein by reference in its entirety as if fully set forth herein.

### FIELD OF THE INVENTION

The invention relates to an infusion cannula unit made of plastic.

### BACKGROUND OF THE INVENTION

Such an infusion cannula unit is known from DE 1 273 749 A, DE 10 2007 055 646 B4 and EP 2 528 642 B1.

### SUMMARY OF THE INVENTION

It is an object of the present invention to further develop an infusion cannula unit having a cannula for insertion in a blood vessel and a housing having a supply line portion which is in fluid communication with a lumen of the cannula, such that production is simplified and, in particular, suitable for mass production.

This object is achieved according to the invention by an infusion cannula unit made of plastic, having a cannula for insertion in a blood vessel, having a housing on which a supply line portion formed the line axis of which runs at an angle relative to a cannula longitudinal axis, said angle being greater than 10°, wherein the supply line portion is in fluid communication with a lumen of the cannula, wherein the infusion cannula unit has a one-piece design.

According to the invention, it has been recognized that it is possible to design the infusion cannula unit in one piece, i.e., in particular, to mold the cannula onto the housing to form one piece. A part assembly of individual parts of the infusion cannula unit, in particular an assembly of the cannula to the housing, is not necessary. In addition, it has been recognized that it is possible to manufacture the cannula, on the one hand, and the housing, on the other, from the same material, so that typical plastic molding techniques are accessible for one-piece manufacture of the unit. The unit can be made from PE and/or from PP.

An infusion cannula unit configured as an injection molded part is particularly suitable for mass production.

A lumen extension in which the cannula lumen widens at the transition into the housing facilitates fluid conduction in the infusion cannula unit.

An infusion cannula unit comprising a conical extension portion is inexpensive to manufacture.

An infusion cannula unit in which the housing has a septum receiving portion results in a simple sealing closure of a housing opening that may remain to be closed due to the manufacturing process.

A metal sleeve which is overmolded by the plastic material of the infusion cannula unit in a cannula region enables a stabilization of the cannula so that it can be made of comparatively soft plastic material. Despite this additional metal sleeve, the unit comprising the cannula and the housing remains configured in one piece. In this case, the metal sleeve is not part of the one-piece unit. In principle, it is also possible to manufacture the cannula entirely from plastic, for example without a metal sleeve.

Examples of embodiments of the invention are explained in more detail below with reference to the drawing.

### BRIEF DESCRIPTION OF THE DRAWING

FIG. 1 shows an infusion cannula unit made of plastic, which is in one piece and has a cannula and a housing connected thereto; and

FIG. 2 shows a further embodiment of a one-piece infusion cannula unit.

### DESCRIPTION OF THE PREFERRED EMBODIMENTS

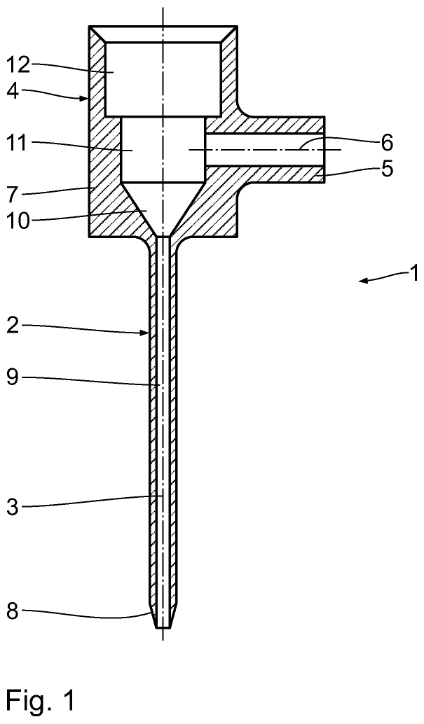

An infusion cannula unit 1 according to FIG. 1 is designed as a one-piece plastic component in the form of an injection molded part.

The infusion cannula unit 1 has a cannula 2 for insertion in a blood vessel of a patient. The infusion cannula unit 1 can be part of an infusion set, in particular for a continuous infusion application.

A cannula longitudinal axis 3 runs vertically in FIG. 1. The infusion cannula unit 1 further includes a housing 4 which is manufactured in one piece together with the cannula 2. A supply line portion 5 is formed on the housing 4 as a likewise one-piece housing component. A line axis 6 of the supply line portion 5 runs horizontally in FIG. 1. The line axis 6 runs at an angle of 90° relative to the cannula longitudinal axis 3. This angle between the line axis 6 and the cannula longitudinal axis 3 is thus greater than 10°. Depending on the design of the infusion cannula unit, the angle between the line axis 6 and the cannula longitudinal axis 3 can be in the range between 10° and 90° and can also be, for example, 30°, 45° or 60°. In these cases, the supply line portion 5 opens in an inclined manner into a base body 7 of the housing 4.

The infusion cannula unit 1 may be made of PE (polyethylene) or PP (polypropylene). Alternatively, the infusion cannula unit 1 may be made of a fluoropolymer or a combination of fluoropolymers, for example PTFE or a thermoplastically processable modification thereof, for example polychlorotrifluoroethylene (PCTFE) or tetrafluoroethylene-hexafluoropropylene copolymer (FEP).

The infusion cannula unit is manufactured by injection molding. An injection mold with drawn cores may be used for this purpose.

The cannula 2 has a conically tapering tip 8. Starting from the tip 8, a cannula lumen 9 initially has a constant inner diameter along the course of the cannula 2. In the base body 7 of the housing 4, the cannula lumen 9 widens via a conical extension portion 10 towards a connecting portion 11, the diameter of which is a multiple of the inner diameter of the cannula lumen 9 in the region of the cannula 2 and, for example, five times this cannula inner diameter. The supply line portion 5 opens into the connecting portion 11.

On the side of the connecting portion 11 opposite the extension portion 10, a receiving section 12 of the housing 4 for a septum not shown in FIG. 1 adjoins the connecting portion for tightly closing the receiving portion 12.

In an embodiment not shown, the infusion cannula unit 1 has a metal sleeve that is overmolded by the plastic material of the infusion cannula unit 1 in a cannula region. Such a metal sleeve may serve to reinforce the cannula 2.

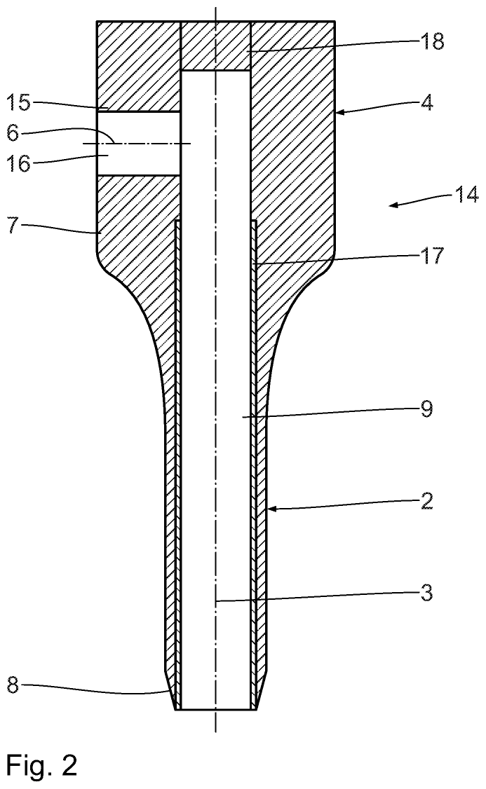

With reference to FIG. 2, a further embodiment of an infusion cannula unit 14 is described below. Components and functions corresponding to those already explained above with reference to FIG. 1 bear the same reference numerals and will not be discussed again in detail.

A supply portion 15 does not protrude radially from a base body 7 of the housing 4 of the infusion cannula unit 14. A fluid passage opening 16 of the supply line portion 15 opens into a cannula lumen 9. In the infusion cannula unit 14, as well, the line axis 6 of the supply line portion 15 has an angle of 90° relative to the cannula longitudinal axis 3. Also indicated in FIG. 2 is a metal sleeve 17, which is overmolded by the plastic material of the infusion cannula unit 14 in the region of the cannula 2.

In FIG. 2, furthermore, a septum 18 for closing an upper end portion of the cannula lumen 9 in FIG. 2.

The infusion cannula units 1 and 14, respectively, are suitable for mass production. In particular, a manufacturing process for these units 1 and 14, respectively, can be made possible in one step.

1. An infusion cannula unit (1; 14) made of plastic,
having a cannula (2) for insertion in a blood vessel,
having a housing (4) on which a supply line portion (5; 15) is formed, the supply line portion (5; 15) extending in a line axis (6) which runs at an angle greater than 10° relative to a cannula longitudinal axis (3), wherein the supply line portion (5; 15) is in fluid communication with a lumen (9) of the cannula (2),
wherein the infusion cannula unit (1; 14) has a one-piece design.

2. The infusion cannula unit according to claim 1, wherein the infusion cannula unit (1; 14) is an injection molded part.

3. The infusion cannula unit according to claim 1, wherein the cannula lumen (9) widens at a transition into the housing (4).

4. The infusion cannula unit according to claim 3, comprising conical extension portion (10).

5. The infusion cannula unit according to claim 1, wherein the housing (4) has a receiving portion (12) for a septum (18).

6. The infusion cannula unit according to claim 1, comprising a metal sleeve (17) which is overmolded by the plastic material of the infusion cannula unit (14) in a cannula region.
